(19) United States
(12) Patent Application Publication
(10) Pub. No.: US 2016/0367580 A1
(43) Pub. Date: Dec. 22, 2016

(54) IMPROVED EXTENDED-ACTION TILMICOSIN AND USE THEREOF IN TREATMENT OF BOVINE RESPIRATORY DISEASE COMPLEX (BRD) AND IN DRY COW PERIOD

(71) Applicant: UNIVERSIDAD NACIONAL AUTONOMA DE MEXICO, Distrito Federal (MX)

(72) Inventors: Hector Salvador SUMANO LOPEZ, Distrito Federal (MX); Lilia GUTIERREZ OLVERA, Distrito Federal (MX)

(21) Appl. No.: 14/890,827

(22) PCT Filed: Feb. 18, 2015
(86) PCT No.: PCT/MX2015/000031

§ 371 Date: Nov. 19, 2015

(30) Foreign Application Priority Data
Feb. 18, 2014 (MX) MX/A/2014/001907

(51) Int. Cl. A61K 31/706; A61K 47/10

(52) U.S. Cl. CPC A61K 31/706; A61K 47/10

(57) ABSTRACT

The invention relates to an improved long-acting or extended-release tilmicosin formulation comprising: tilmicosin phosphate in a concentration between 35 and 50% by weight of the total composition; a first co-solvent in a concentration between 8 and 20% by volume, said first co-solvent being propylene glycol; a second co-solvent in a concentration between 5 and 15% by volume, said second co-solvent being ethyl alcohol; and an emulsifier in a concentration between 1.5 and 15% by weight of the total composition, said emulsifier being poloxamer. The invention also relates to the method for producing said improved long-acting tilmicosin formulation.

### FIELD OF INVENTION

The present invention is related to principles and techniques used in veterinary pharmaceutical industry for developing new pharmaceutical compositions for drug manufacturing which contribute to animal health, and more specifically is related to a formulation comprising improved extended-action tilmicosin, a method for manufacturing thereof and its use in prevention and treatment of Bovine Respiratory Disease Complex (BRD) and during the dry cow period.

### BACKGROUND OF INVENTION

Bovine cattle industry represents one of the important economic activities in the country. However, productivity thereof has decreased in recent years and one of the cause factors are the limited sanitary activities conducted at the cattle units. In case of bovines, there are a number of diseases affecting them, being remarkable in this regard the so-called “Bovine Respiratory Disease Complex” (BRD) a disease which causes large economic losses. The economic impact is not only based on morbidity and mortality, but is also due to a reduction in productive variables of bovines specialized in milk and meat production and dual purpose.

Main losses caused by BRD are: medication expenses for diseased animals, maintenance of cattle under recovery, decrease in daily weight gain, involvement of final yield, decrease in meat and milk production and cattle death.

It has been determined that respiratory disorders in bovines are due to multifactorial causes where two groups of factors coexist or interact: predisposing factors and determining or triggering factors. Predisposing or stressing factors include a number of factors related to management and environment such as transportation, overcrowding and deficient feeding wherein said adverse environmental conditions create optimal conditions for bacterial proliferation. On the other hand, determining causes are referred to viral and bacterial infectious agents that ultimately trigger the clinical condition.

Several viruses participate in BRD such as Bovine Infectious Rhinotracheitis (IBR) virus, Bovine Viral Diarrhea (BVD), Parainfluenza 3 (PI3) and Bovine Syncytial Respiratory Virus (VRSB), damaging the respiratory system and predisposing to bacterial infections. Bacteria which are commonly involved are Type A1 Pasteurella haemolytica and its leukotoxin, Pasteurella multocida and Haemophilus somnus (currently Hitophilus somni), said agents which are normally identified in the respiratory tree as a final cause of pneumonia and death in bovines.

As BRD is one of the three main causes of cattle disposal, its timely diagnosis and treatment is relevant to prevent death and respiratory problem chronicity leading to decrease in animal production, drug expenses and medical costs as well as animal death.

BRD treatment is oriented towards symptomatology present in any animal by a combined use of therapeutic agents acting as mucolytics, bronchodilators, anti-inflammatory and antibacterial agents empirically selected or through an antibiogram. In mild cases, a minimum treatment by 3 days solves the clinical condition while the treatment with antibiotics results commonly inefficient in severe cases of pneumonia with foci of consolidation. In proved cases of BRD where a virus-bacteria interaction is present, the essential subject is a control by using bacterines and/or vaccines allowing reaching good protection in bull calves in the critical stage from birth until 3 months, and methaphylactic treatments are often applied.

On the other hand, it is known that milk producing cattle worldwide requires a rest period before labor and before starting a new lactation period, wherein such rest period is known as “dry cow period” or “dry period”. This “dry cow period” is useful to recover wasting due to lactation which is left behind and then supply sufficient metabolic reserves (including Ca, P, Mg, etc.) for labor and offspring breastfeeding. During this stage the animal is expected to recover the mammary tissue and rests to reach a proper development of later lactation in addition to enhance fetus growth.

Said “dry cow period” lapses from 30 to 60 days prior to labor and the more used period worldwide is 45 days and treatments provided in this period are known as drying therapy. Stress is present in the cow during “dry period” commencement and towards the completion thereof. Upon commencement the cow can no longer be milked and there is stress by routine loss and intramammary pressure during milk material reuptake as well as towards the end of said “dry period”.

Similarly, Gram-positive bacteria, particularly Staphylococcus aureus, may cause clinical and/or subclinical mastitis during the “dry period” commencement. During dry period end period Streptococcus uberis is more commonly present as the more common cause of infection while Gram-negative bacteria infections are very uncommon.

Above mentioned clinical and specifically subclinical infections generate clinical mastitis during the first 8 weeks of lactation and then incidence tends to be lower. In order to control such situation, antibacterial medicament intramammary tubes are administered. It is known that this must be done only once before starting the “dry period” and it is intended that the preparation lapses the longest possible time within the gland in said “dry period”. However, the following disadvantages are identified for antibacterial treatment in “dry period” by intramammary route:
Permanence time of preparations is not known since milk samples may not be obtained as this would interrupt the “drying” physiological process.
Many preparations have a poor pharmaceutical design in terms of diffusion to mammary tissue from the mammary cisternal.
Intramammary preparations shall be applied with aseptic technique intending not to lacerate the teat channel.
If a reinforcement were required during “dry period” end stage, intramammary application would damage more than what would help.

Feeding and handling during the “dry cow period” or “dry period” have been the more careless issues in milk cow herds for a long time. Nowadays it is known that a bad management or deficient feeding would derive in a great amount of metabolic disorders which are manifested as diverse pathological problems such as downer cow syndrome, milk fever, ketosis, displaced abomasum, placental retention, laminitis, labor difficulty, productive difficulty due to wrong estrus, production decrease, and the like.

For treatment of bacterial-type respiratory infectious diseases, Mexico has available the following options as to antibiotics:

a) Florfenicol+flunixin-meglubine which is subcutaneously administered (SC) at a dose of 40 mg/kg and 2.2 mg/kg florfenicol and flunixin, respectively and which according to the manufacturer reaches therapeutic concentrations for 96 hours. Only 1 dose and exceptionally 2 doses are recommended.

b) Tilmicocin at a 10 mg/kg dose subcutaneously (SC) in single dose, a maximum of two with an interval of 72 hours.

c) Crystallized ceftiofur, subcutaneously administered (SC) at ear basis contributing to a better meat quality. Doses are from 14 mg/kg with a useful concentration from 7 to 10 days.

d) Enrofloxacin-LA, which is applied at a dosage from 7.5 to 10 mg/kg achieving therapeutic concentrations up to 72 hours according to the manufacturer.

e) Tulathromycin which is a new extended action macrolide. It is an injectable sterile antimicrobial solution ready to be used in a single dose comprising 100 mg of tulathromycin/ml in propylene glycol aqueous formulation. It allows dosage intervals from 14 to 21 days after its subcutaneous route (SC) application. It is administered on neck at a 2.5 mg tulathromycin/kg body weight dose.

With specific reference to tilmicosin, it is a macrolide antibiotic synthesized from tylosin, which has been available in United States since 1992 and exclusively developed to be used in veterinary medicine.

Macrolide antibacterial activity is produced by inhibition of protein synthesis by binding the antibiotic in the prokaryote microorganism ribosomal 50S subunit ARN23S. Macrolides inhibit transfer RNA translocation from amino acid acceptor point which prevents forming a new peptide bind thus preventing new protein synthesis in microbial cell. Macrolides are bound to mitochondrial ribosomes but they are unable to traverse mitochondrial membrane and therefore do not elicit bone marrow suppression in mammals.

Tilmicosin has in-vitro activity against gram-positive organisms and mycoplasmas and is active against certain gram-negative organisms, such as Histophilus sommi (Haemophilus somnus), Mannheimia haemolytica and Pasteurella multocida and Pasteurella haemolitica. Macrolide antibacterial activity is produced by protein synthesis inhibition by binding the antibiotic in the prokaryote microorganism ribosomal 50S subunit ARN23S. Moreover, macrolide exert a significant anti-inflammatory and immunomodulatory activity.

There are a number of documents related to tilmicosin within the state of the art, such as Chinese Patent No. CN 103083281 (A) referred to the preparation of a slow-release tilmicosin microcapsule with enteric coating and to a method of preparation thereof which belongs to tilmicosin preparations. Preparation of a slow-release enteric coating tilmicosin microcapsule provided by the invention comprises an inner core layer and a coating layer, wherein the inner core layer comprises powdered tilmicosin and an ancillary material; the ancillary material comprises one or more than one of stearic acid, glycerin monostearate, stearyl alcohol, saturated triglyceride, monoglyceride and paraffin, and the coating layer is made of one or more than one of cellulose acetate phtalate, hydroxypropyl-methyl cellulose phtalate, acrylic resin, polyvinyl phtalate acetate and hydroxypropyl methylcellulose succinate acetate. The preparation method comprises the following steps: performing a primary coating over tilmicosin powder and ancillary material; performing a second coating by using coating layer materials and drying to obtain the final product. According to the invention, tilmicosin is coated by using high polymer materials and the coated tilmicosin microcapsule is non-soluble in acidic means and the enteric coating is slowly dissolved in alkaline means such that the purpose of slow release is achieved and tilmicosin action time is extended.

On the other hand, Chinese patent No. CN101249069 (A) refers to the technology field of veterinary drug preparation for poultry and cattle, specifically to an extended-release tilmicosin injectable preparation and a preparation method thereof. Availability of tilmicosin preparations (such as aqueous solutions, tablets and common injections) having a weight gain effect in treatment group chickens at high tilmicosin doses and treatment groups at mean tilmicosin dose has been currently reported, but they do not show any significant difference with tylosin treatment groups, and tilmicosin is better than tylosin at lower dose and a remarkable anti-infectious effect. The invention provides an extended-action tilmicosin injection and a preparation method thereof. The extended-action tilmicosin injection is prepared by dissolving the bulk tilmicosin drug in anhydrous alcohol and castor oil, eventually mixing, sub-packaging and sterilizing. The extended-action tilmicosin injection has the advantages of a single, low-cost preparation process and a remarkable healing effect, and is a new veterinary medicament for poultry and cattle, with high safety and efficacy, low price and less toxicity.

Chinese patent No. CN101416978 (A) also describes a method of preparation of a long-action tilmicosin injection comprising the following steps: (1) 10 g to 20 g of tilmicosin are accurately weighed and put into a beaker; (2) 25 ml to 75 ml de propylene glycol are added into a graduated cylinder and poured into tilmicosin, stirring, double boiling and dissolving completely at a temperature of 60° C., and then water is added which is used in injections; (3) pH value is regulated with phosphoric acid to be from 5.5 to 6.5; (4) volume is taken to 100 ml, and (5) after filtering and encapsulating, sterilization is carried out at a temperature of 100° C. during 30 min, and the final product is obtained after cooling, slightly inspecting, printing and packaging. The veterinary medicament has advantages of advanced production techniques, stable preparation, long storage life, quick effect, long low half-life period from 2 days to 3 days, a safe use, exact therapeutic effect, suitable applications and the like.

As seen from the state of the art, a number of drugs are known to prevent and to treat bacterial-type infectious diseases; however, all those show certain drawbacks such as:

Tulathromycin: its antibacterial potency is reduced upon decreasing pH in an already infected respiratory system. Therefore, it is more frequently used as preventive agent.

Florfenicol LA and LA improved: A second and even a third application often required in the first case thus leading to a very difficult handling in large fattening; while in the second case even when plasma concentrations last up to 120 hours, formulation viscosity makes it difficult to apply, especially when related to large size extensions.

Enrofloxacin: there are a number of very low quality generic drugs which are not bioequivalent. This drug often requires more than one dose and is not used preventively, but only as treatment.

Acidic crystal ceftiofur: This requires a special injection technique on ear and is only useful for treatment.

Oxitetracycline LA: Already showing a lot of resistance and requiring several doses when used as preventive.

### BRIEF DESCRIPTION OF INVENTION

It has been surprisingly found that by increasing the therapeutic effect time of tilmicosin the efficiency is significantly improved in treatment and/or prophilaxis of bacterial type infectious diseases of respiratory system, preferably in treatment of BRD and further achieving a therapeutic and preventive effect of intramammary infections since parenteral application, in the period of “dry cows” and accordingly during the first lactation stages (being those more susceptible).

In the light of above, a new tilmicosin composition was developed, formulated for improved extended release or action, in the range from 9 to 10 days in plasma regarding reference tilmicosin which only shows a range from 2 to 3 days, in addition that said new tilmicosin formulation does not show cardiotoxic effects.

Therefore, the present invention is referred to said new improved tilmicosin composition formulated for extended action or release comprising: tilmicosin phosphate in a concentration from 35 to 50% by weight of total composition; a first co-solvent in a concentration from 8 to 20% by volume, wherein said first co-solvent is propylene glycol; a second co-solvent in a concentration from 5 to 15% by volume, wherein said second co-solvent is ethyl alcohol; and, an emulsifier in a concentration from 1.5 to 15% by weight of total composition, wherein said emulsifier is poloxamer.

The extended-action improved tilmicosin described in accordance with a particularly preferred embodiment of present invention has the following formula:

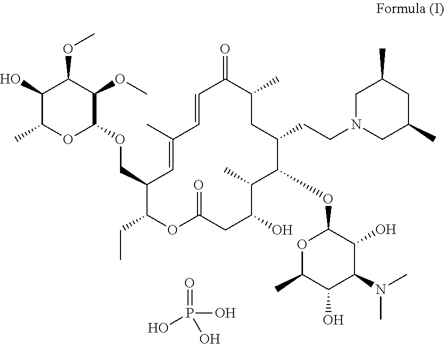

On the other hand, the present invention provides a method for obtaining an improved extended-action tilmicosin described and claimed in present invention, wherein said method comprises the steps of:

(a) Transferring from 45 to 60 ml, preferably 53 ml of distilled water to a beaker:

(b) Adding to the beaker containing distillate water an amount from 8 to 20 ml of a first co-solvent which is propylene glycol, and stirring until obtaining a homogeneous solution.

(c) Adding an amount from 5 to 15 ml of a second co-solvent being ethyl alcohol, and covering the beaker with parafilm paper and mixing until obtaining a homogeneous solution.

(d) Introducing an amount from 35 to 50 g of tilmicosin phosphate, slowly in order to prevent lump formation, and stirring until obtaining a homogeneous solution.

(e) Adding from 1.5 to 15 g of an emulsifier, wherein said emulsifier is poloxamer, making a slow addition to achieve solution homogenization.

(f) Refrigerating above solution at a temperature from 2 to 8° C. during 1 hour; removal from refrigeration and stirring for 5 minutes.

(g) Refrigerating again for 24 hours stirring until obtaining a full poloxamer disolution, obtaining the final product.

### OBJECTS OF THE INVENTION

Taking into account the defects of prior art, it is an object of present invention to provide a new tilmicosin formulated for improved extended action or release.

It is a further object of present invention to provide a new improved extended-action tilmicosin formulation, lapsing from 9 to 10 days in plasma and at least 20 days at tissue level both in respiratory system and in the mammary gland.

A further object of present invention is to provide a new improved extended-action tilmicosin formulation useful in treatment and prophylaxis of bacterial-type respiratory infectious diseases, preferably in treatment of Bovine Respiratory Disease Complex (BRD) and in prophylaxis and treatment of milk producing cows within the period “dry cows”.

It is still another further object of present invention to provide an improved extended-action tilmicosin base pharmaceutical composition for manufacturing a drug used in veterinary medicine against bacterial type respiratory infections, as well as in prophylaxis and treatment of mammary gland in milk producing cows in the so-called period of “dry cows”.

Still a further object of present invention is to provide an improved extended-action tilmicosin base pharmaceutical composition not having cardiotoxic effects.

Still a further object of present invention is to provide a method for preparation of improved extended-action tilmicosin.

### BRIEF DESCRIPTION OF FIGURES

Novel aspects which are considered characteristic of present invention shall be particularly established in the attached claims. However, the invention itself both as to its organization and its method of operation, together with other objects and advantages thereof, is better understood in the following detailed description of a particularly preferred embodiment of the present invention, when read in relation to the attached drawings wherein:

FIG. 1 is a basal ECG tracing and 2 hours after application of the improved extended-action tilmicosin preparation [(Til_{APM} (1)] at a dose of 24 mg/kg.

FIG. 2 is a basal ECG tracing and 2 hours after application of the improved extended-action tilmicosin preparation [(Til_{APM} (2)] at a dose of 30 mg/kg.

FIG. 3 shows urea, creatinine and albumina mean concentrations in animals medicated with Til_{APM} (1) and Til_{APM} (2) before administering this preparation (basal value on day zero) and 7, 14 and 21 days after.

FIG. 4 shows values of plasma CPK after administering Til_{APM} (1) and Til_{APM} (2), as well as reference tilmicosin [(Til_{REF} (Micotil®)] at a dose of 10 mg/kg.

FIG. 5 is a graph where recovery curve regression used to quantify the tilmicosin activity/concentration in serum is shown (r²=0.98).

FIG. 6 is a graph where ±1 SD average of tilmicosin phosphate (improved extended-action tilmicosin) preparation concentrations is shown and is compared with data achieved for reference tilmicosin (Micotil®).

FIG. 7 is a graph where kinetic profiles of dual reference tilmicosin doses (Micotil®) from a 10 mg/Kg injection are shown.

FIG. 8 is a graph where tilmicosin concentrations in milk or milk fluid extracted during dry period of Holstein/Friesian cows, after two tilmicosin administrations of Til_{APM} (1) at 24 mg/kg dose on day 0 and on day 20 are shown.

FIG. 9 is a graph where the number of post-labor mastitis cases per week is shown, in cows treated upon starting dry period with various intramammary antibiotics and registered for such use (conventional treatment) and cows parenterally treated with Til_{APM} (1) at 24 mg/kg dose upon starting dry period and subcutaneously (SC) 20 days later.

FIG. 10 is a graph where count trends of somatic cells in milk during 8 weeks after labor are shown, proceeding from cows treated upon starting dry period with various intramammary antibiotics and registered for such use (conventional treatment), and cows treated parenterally with Til_{APM} (1) at 24 mg/kg dose upon starting dry period and subcutaneously (SC) 20 days later.

FIG. 11 shows calibration curve type chromatograms obtained from HPLC reading.

FIG. 12 is a graph showing a tilmicosin base calibration curve obtained from HPLC reading.

FIG. 13 shows type chromatograms obtained by HPLC from fortified liver samples with base tilmicosin at a concentration of 10 ng/ml.

FIG. 14 shows type chromatograms obtained by HPLC from fortified kidney samples with base tilmicosin at a concentration of 10 ng/ml.

FIG. 15 shows type chromatograms obtained by HPLC from fortified muscle samples with base tilmicosin at a concentration of 10 ng/ml.

FIG. 16 shows type chromatograms obtained by HPLC from fortified fat samples with base tilmicosin at a concentration of 10 ng/ml.

FIG. 17 is a graph showing the tilmicosin base concentrations in several studied tissues.

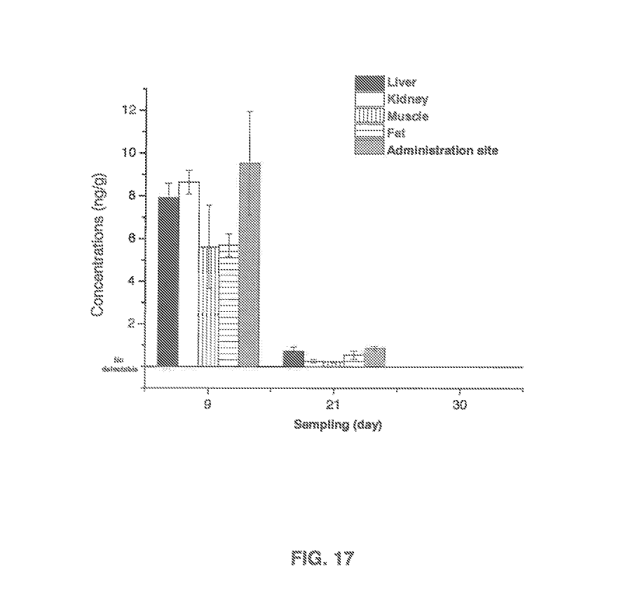

### DETAILED DESCRIPTION OF INVENTION EMBODIMENTS

It has been surprisingly found that by increasing the therapeutic effect time of tilmicosin, efficiency in treatment and/or prophylaxis of bacterial-type respiratory infectious diseases is strongly improved, preferably in treatment of BRD and in the “dry cow” period.

In the light of that above, a new tilmicosin composition formulated for improved extended-release or action was developed, ranging from 9 to 10 days in plasma compared to reference tilmicosin having only a range from 2 to 3 days, in addition that said new tilmicosin formulation does not have cardiotoxic effects.

Therefore, the present invention refers to said new tilmicosin composition formulated for improved extended action or release comprising: tilmicosin phosphate in a concentration from 35 a 50% by weight, preferably 42% by weight of total composition; a first co-solvent in a concentration from 8 to 20% by volume, preferably 10% by volume, wherein said first co-solvent is selected from the group comprising the more common co-solvents used in drug formulation such as sorbitol, glycerin, glyceride ester derivatives, ethyl alcohol, isopropyl alcohol, propylene glycol, benzyl alcohol, low molecular weight polyethylene glycol (100, 200, 300, 400, 600 and 800) and polyethylene glycol derivatives, dimethyl sulfoxide, glycerol formal, glycofurol, ethyl carbonate, ethyl lactate, dimethyl acetamide or 2-methyl pyrrolidone, and the like; preferably using propylene glycol; a second co-solvent in a concentration from 5 to 15% by volume, preferably 10% by volume, wherein said second co-solvent is selected from the group comprising the more common co-solvents used in drug formulation such as sorbitol, glycerin, glyceride ester derivatives, ethyl alcohol, isopropyl alcohol, propylene glycol, benzyl alcohol, low molecular weight polyethylene glycol (100, 200, 300, 400, 600 and 800) and polyethylene glycol derivatives, dimethyl sulfoxide, glycerol formal, glycofurol, ethyl carbonate, ethyl lactate, dimethyl acetamide or 2-methyl pyrrolidone, and the like; using ethyl alcohol; and an emulsifier in a concentration from 1.5 to 15% by weight, preferably 3% by weight of total composition, wherein said emulsifier may be any surfactant with emulsifying activity capable of stabilizing the system, preferably using poloxamer as emulsifier.

For the purposes of present description, controlled release shall be understood as any formulation technique wherein active principle or drug release from the dose form is modified to occur at a slower rate than that from an immediate release product and then the concentration of said drug is maintained for an extended time.

Also, retarded release is understood as any formulation technique wherein the active substance or drug release from the dosage form is modified to occur at a time later than an immediate release conventional product. Subsequent release of active substance from a retarded release formulation may be also controlled as previously defined.

The improved extended-action tilmicosin described in accordance with a particularly preferred embodiment of present invention has the following formula:

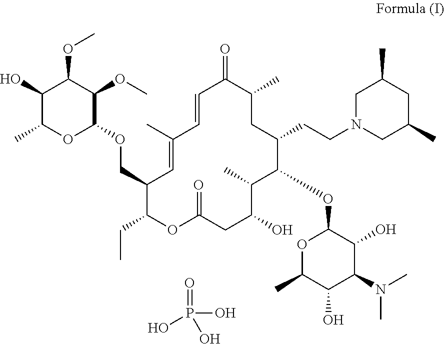

On the other hand, the present invention provides a method to prepare the improved extended-action tilmicosin described and claimed in present invention, wherein said method comprises the steps of:

(a) Transferring from 45 to 60 ml, preferably 53 ml, of distilled water to a beaker:

(b) Adding to the beaker containing the distilled water an amount from 8 to 20 ml, preferably 10 ml, of a first co-solvent selected from the group comprising more common co-solvents used in drug formulation such as sorbitol, glycerin, glyceride ester derivatives, ethyl alcohol, isopropyl alcohol, propylene glycol, benzyl alcohol, low molecular weight polyethylene glycol (100, 200, 300, 400, 600 and 800) and polyethylene glycol derivatives, dimethyl sulfoxide, glycerol formal, glycofurol, ethyl carbonate, ethyl lactate, dimethyl acetamide or 2-methyl pyrrolidone, and the like; preferably using propylene glycol and stirring until obtaining a homogeneous solution.

(c) Adding an amount from 5 to 15 ml, preferably 10 ml, of a second co-solvent selected from the group comprising the more common co-solvents used in drug formulation such as sorbitol, glycerin, glyceride ester derivatives, ethyl alcohol, isopropyl alcohol, propylene glycol, benzyl alcohol, low molecular weight polyethylene glycol (100, 200, 300, 400, 600 and 800) and polyethylene glycol derivatives, dimethyl sulfoxide, glycerol formal, glycofurol, ethyl carbonate, ethyl lactate, dimethyl acetamide or 2-methyl pyrrolidone, and the like; preferably using ethyl alcohol. Close the beaker with parafilm paper and mixing until obtaining a homogeneous solution.

(d) Slowly introducing an amount from 35 to 50 g, preferably 42 g, of tilmicosin phosphate, in order to prevent lump formation, and stirring until obtaining a homogeneous solution.

(e) Adding an amount from 1.5 to 15 g, preferably 3 g, of an emulsifier, wherein said emulsifier may be any surfactant with emulsifying activity capable of stabilizing the system, preferably using poloxamer as emulsifier, making a slow addition in order to achieve solution homogenization.

(f) Refrigerating above solution at a temperature from 2 to 8° C. during 1 hour. Removing from refrigeration and stirring by 5 minutes.

(g) Refrigerating again for 24 hours stirring until obtaining complete poloxamer dissolution, thus obtaining the final product.

A specific aspect of present invention is to provide an improved extended-action tilmicosin base pharmaceutical composition used in veterinary medicine. Moreover, the use of said pharmaceutical composition for manufacturing a medicament useful in treatment of bacterial-type respiratory infectious diseases such as Bovine Respiratory Disease Complex (BRD) and the “dry cow” period and the like, is provided.

The present invention will be better understood from the following examples, which are only presented for illustrative purposes allowing a full understanding of the embodiments of present invention, without implying that there are no other embodiments not illustrated which may be put into practice based on above disclosed detailed description.

It is worth to mention that for a better description of the examples, the improved extended-action tilmicosin described and claimed in the preferred embodiment of the present invention has been identified as Til_{APM} and reference tilmicosin as Til_{REF}.

### Example 1

### Safety Testing Protocol for Improved Extended-Action Tilmicosin (Til_{APM})

Longer term plasma concentrations are achieved with Til_{APM}, thus improving its pharmacokinetics/pharmacodynamics ratio.

Tilmicosin was chosen because it is one of the first choice drugs for treatment of BRD. It shows an increased distribution even penetrating at intracellular level and showing less bacterial resistance compared to 14-atom macrolides. It shows anti-inflammatory and immunomodulating properties and is useful against a wide range of pathogens such as: (Mannheimia haemolytica, Pasteurella multocida, Histophilus somni, Mycoplasma spp)

An optimal concentration is intended along a single dose or instead, during the treatment interval, taking into account that macrolides have excellent distribution towards lung tissues and they are considered time-dependent drugs from a clinical point of view. Based on literature findings, plasma concentration targets are established to fluctuate between 0.1 and 2 μg/ml. It is particularly important for tilmicosin not to exceed 4 μg/ml concentrations since tilmicosin is known to induce cardiotoxicity with tachycardia. The challenge of achieving said concentrations is reached by preventing peak values in plasma (cardiotoxic), in this case even increasing the dose at potentially toxic levels of 24-30 mg/kg. These doses include that indicated for the new prototype and any margin due to miscalculation in bovine weight.

On such background, an improved sustained-release tilmicosin parenteral formulation was designed which shall be delimited between such limits in order to prevent cardiotoxic effects. An improved extended-release or extended-action preparation has significant therapeutic advantages as it is known that a time-dependent medicament, and particularly for managing meat-producing bovines, represents obvious advantages such as stress decrease by handling a second injection, manpower and equipment savings but above all, a better clinical efficacy is established.

Material and Methods

Location and Animals:

This stage was conducted at CEIEPASP facilities. 21 randomly-selected bull calves were used in 4 groups of 8 bovines each reference group [(Til_{REF} (Micotil®)] and Til_{APM} treated groups. Animals were kept without antibacterial treatment or of any other nature at least 30 days in advance. Schedules for deparasitation and immunization were kept according to farm management practices. Dosage was performed as follows:

Reference Group (R Group):

Each bovine (8 in total) was weighed and tilmicosin was individually dosed subcutaneously (SC) at 10 mg/kg of original preparation (Til_{REF}) without applying more than 10 ml per application site, spreading the dose on the back and on flabby part of the neck.

Overdosed Reference Group (Rsd Group):

Each bovine (8 in total) was weighed and individually dosed with tilmicosin by SC route at 20 mg/kg of original preparation (Til_{REF}) without applying more than 10 ml per application site, spreading the dose on the back and flabby part of the neck.

Group Til_{APM} (1):

A dose of 24 mg/kg to each bovine (8 in total). They were weighed and dosed individually with tilmicosin SC at a 24 mg/kg rate of Til_{F} preparation without applying more than 10 ml per application site, spreading the dose on the back and flabby part of the neck.

Til_{APM} Group(2):

One dose of 30 mg/kg to each bovine (8 in total). They were weighed and individually dosed with tilmicosin by SC route at 30 mg/kg of Til_{REF} preparation without applying more than 10 ml per application site, spreading the dose on the back and flabby part of the neck.

Follow-ups for heart frequency, respiratory frequency and ECG trace were then conducted by means of an ECG Fukuda cardisuny 501B-III®, putting percutaneous needle electrodes wetted with ethanol at 70%. ECG logs in derivate II were taken at the following times: 1, 4, 8 and 24 hours post drug administration when tilmicosin Cmax occurs.

Furthermore, basal blood samples were taken (hour zero) and on days: 1, 3, 5, 7, 9 and 11 after Til_{APM} (1) and Til_{APM} (2) dosage, as well as with reference group (Group R), in order to determine CPK profiles (CPK ATP-cysteine 334 nm, Wiener) in plasma as measure of muscle damage adjacent to injection(s) and possible cardiotoxicity.

Blood samples were also taken on 7, 14 and 21 days to determine variables related to kidney profile (urea, creatinine and albumina). Moreover, all animals were daily observed during the period of study in order to detect any sign that would show any adverse reaction or toxicosis, such as anaphylaxis, tearing, salivation, diarrhea, liver toxicosis, nephrotoxicosis, hemoglobinuria, inflammatory reaction on administration site and photosensitization.

Results:

Til_{APM} (1) and Til_{APM} (2) were not detected in R groups neither tachycardias nor inversions in T wave, being characteristic changes of overdosing. An increase in heart frequency was detected in all animals within Rsd group of a mean of 36±4.8 beats per minute at 48±6.4 beats per minute. However, this effect only remained for about 8 hours and it was not observed in any animal after this time.

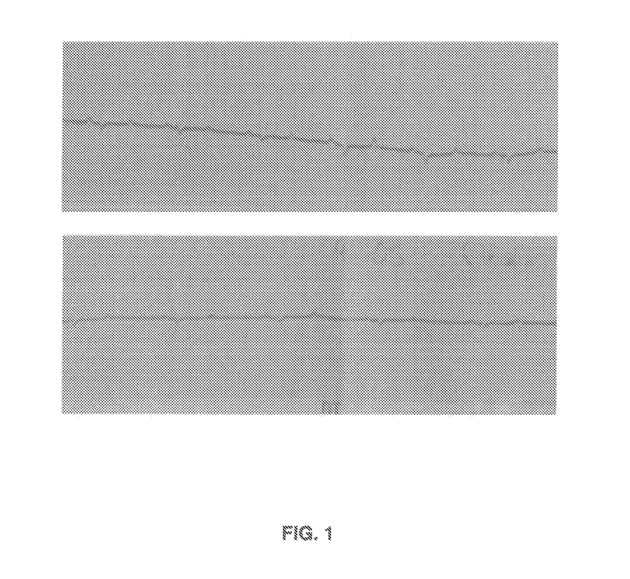

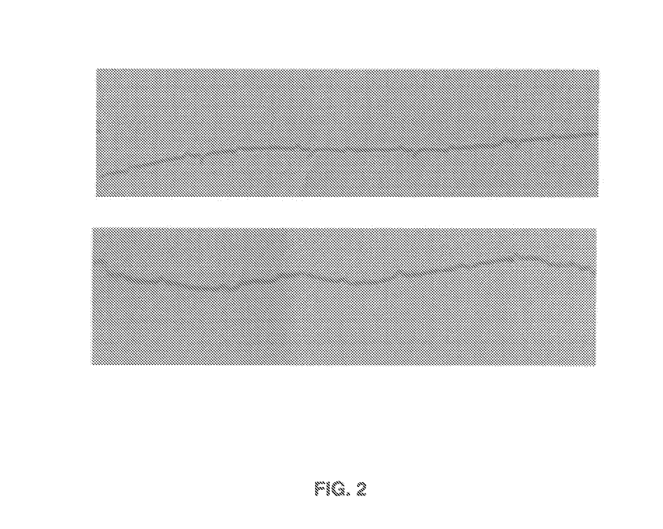

FIGS. 1 and 2 of attached drawings show ECG traces typical of groups Til_{APM} (1) and (2), respectively.

Table 1 shows the clinical response in terms of breathing and heart frequency upon subcutaneous (SC) application of Til_{APM} (2) at a dose of 30 mg/kg. As noticed, substantial changes were not detected in measured variables.

TABLE 1
Ratio of heart frequencies with two dose types of
Til_{REF} preparation and TIL_{APM} preparation subcutaneously
injected (SC) in bovines.

| | | Basal | | | | | |
|---|---|---|---|---|---|---|---|
| | Tilmicosin | heart | | | | | |
| | dose | frequency | Post-injection heart frequency (X ± SD) | | | | |
| GROUP | mg/kg | X ± SD | 30 min | 1 hr | 2 hr | 4 hr | 12 hr |
| Reference | 10 | 41 ± 10 | 46 ± 6.8 | 47 ± 7.5 | 42 ± 8.5 | 40 ± 6.5 | 40 ± 8.5 |
| Group | | | | | | | |
| Over- | 20 | 42 ± 8.5 | 49 ± 8.9 | 49 ± 10.5 | 43 ± 9.5 | 41 ± 8.4 | 40 ± 6.4 |
| dosed | | | | | | | |
| Reference | | | | | | | |
| Group | | | | | | | |
| Group | 24 | 39 ± 9.8 | 46 ± 8.4 | 48 ± 9.5 | 44 ± 10.5 | 40 ± 5.8 | 40 ± 8.8 |
| Til_{APM} (1) | | | | | | | |
| Group | 30 | 40 ± 6.5 | 47 ± 8.8 | 49 ± 6.8 | 45 ± 8.5 | 40 ± 7.8 | 40 ± 9.8 |
| Til_{APM} (2) | | | | | | | |

### No Statistically Significant Differences were Detected Between Basal Values and Values after Application of Tilmicosin Formulations

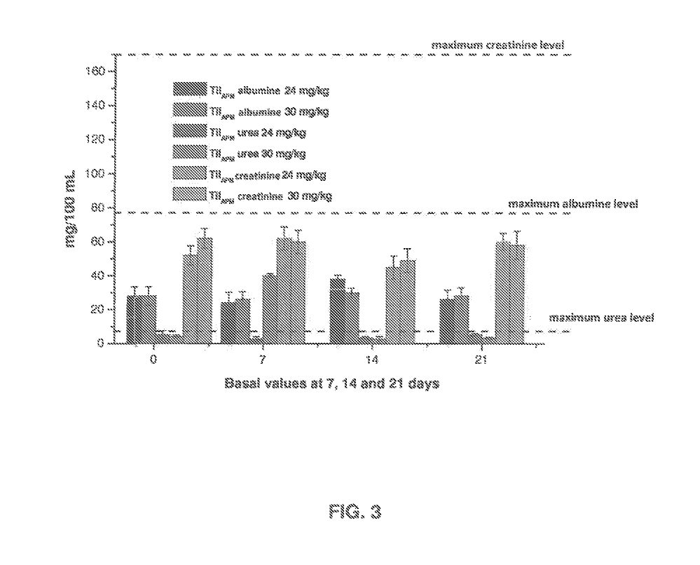

Moreover, urea, creatinine and albumina mean concentrations in medicated animals with improved extended-action tilmicosin are shown in FIG. 3, at doses of 24 mg/kg [(Til_{APM} (1)] and improved extended-action tilmicosin at doses of 30 mg/kg [(Til_{APM} (2)] before administering this preparation (basal value on day zero) and 7, 14 and 21 days later, applying no more than 10 ml per site.

CPK values were momentarily raised during the first day due to moderate irritation on application site and similarly to that observed with almost any medicament.

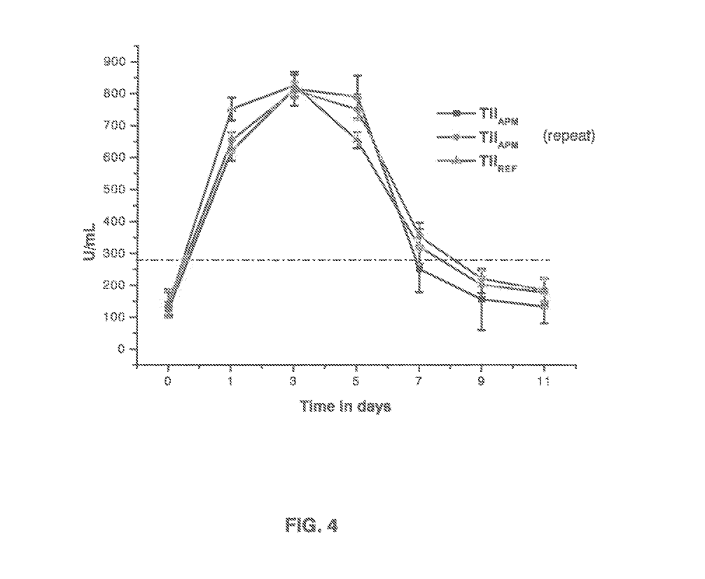

FIG. 4 of attached drawings shows this trend for groups Til_{APM} (1), Til_{APM} (2) and Til_{REF} at a dose of 10 mg/kg. It is important to remark that slight inflammation on administration sites was present in the three groups, not lasting more than 72 hours in any case. In fact that disappeared mostly after 48 hours in agreement with a drop in normal values in CPK values. Moreover, it is important to remark that there is a full recovery in all cases in the concentrations of these enzymes.

Conclusions:
There are no adverse reactions at administration site of Til_{APM} (1) or Til_{APM} (2) in volumes which are never higher than 10 mL per application site.
Side effects were not detected upon clinical examination in any of the bovines included in this trial in the 4 groups.
Significant or important variations in heart frequency or in ECG trace suggesting heart toxicity were not detected in experimental groups of Til_{APM} (1) and Til_{APM} (2).
Changes in plasma CPK concentration were detected with no more than 7 days in Til_{APM} (1) and Til_{APM} (2) groups, as well as of Til_{REF} (Micotil®) at dose of 10 mg/kg, compatible with the inflammatory process observed and typical for injected macrolides and without any relation to a possible heart toxicity since changes in heart frequency and ECG trace were not detected.
No significant or important changes were detected in variables defining kidney function: urea, creatinine and albumina in serum.

### Example 2

### Pharmacokinetic Study of Improved Extended-Action Tilmicosin (TIL_{APM}) in Bovines

Animals.—

45 bull calves randomly distributed in 3 groups of 7 bovines each were used. Group 1 was treated with Til_{APM}(A), treated group 2 Til_{APM}(B) (repeat of test A) and group 3 with reference tilmicosin Til_{MFF} (Micotil®). Animals were kept without antibacterial treatment or any of another nature at least 30 days in advance. Schedules of deparasitation and immunization were kept in accordance with farm management practices.

Dosage was performed as follows:

Reference Group 3:

Each bovine was weighed and dosed individually subcutaneously (SC) with tilmicosin at a 10 mg/kg ratio of original preparation (Elanco's Micotil®), without applying more than 10 ml per application site, spreading the dose on the back and flabby part of the neck.

Bleeding was performed at the following times after administration: 15 and 30 minutes, 1, 1.5, 2, 2.5, 4, 8, 12, 24, 36, 48, 72, 96 and 120 hours, obtaining a minimum of 10 ml per bleeding time. Samples were collected by jugular vein puncture with vacutainer tubes. Blood samples were centrifuged at 3000 rpm during 10 minutes, serum was separated and samples were kept in freezing (−4° C.) until the time of analysis, which was carried out before 1 month.

Heart frequency, respiratory frequency and electrocardiographic trace was monitored by means of an ECG Fukuda cardisuny 501B-III®, placing percutaneous needle electrodes wetted with ethanol at 70%. ECG logs in derivative II were taken on the following times: 1, 4, 8 and 24 hours post drug administration.

Group 1:

Each bovine was weighed and dosed with Til_{APM}, at a dose of 24 mg/kg (1 ml/15-20 kg of weight) and blood samples were obtained on above reported times in group 3. Blood samples were collected and handled in the way already disclosed, as well as monitoring with ECG.

Group 2 (Repeat):

Each bovine was weighed and dosed with Til_{APM}, at a 24 mg/kg dose (1 ml/15-20 kg of weight) and blood samples were obtained in above reported times. Blood samples were collected and handled as already disclosed, as well as monitoring with ECG.

All animals were daily observed during study period in order to detect any sign which would show any adverse reaction or toxicosis, such as anaphylaxis, tearing, salivation, diarrhea, liver toxicosis, nephrotoxicosis, hemoglobinuria, inflammatory reaction on administration site and photosensitization.

Tilmicosin concentrations were determined by an analytical technique of plate diffusion (which is described below) and pharmacokinetic runs were performed with individual concentrations using the program WinNonline (WinNonlin Version 3.2, Pharsight, Mountain View, Calif., USA) and PkAnalyst (MicroMath, Salt Lake, Utah, USA) and model 3 to determined its compartmental pharmacokinetics by the following general formula:

$$Concentration ( Time ) = \frac{Dose * {K}_{AB}}{Volume {K}_{AB} - {K}_{elim}} e {-}^{{K}_{elim}} * Time - e {-}^{{K}_{AB}} * Time$$

The following variables were determined: AUC_{0-∞}; AUMC; MRT; α; b; Kₑₗ; T_{1/2ab}; T_{1/2b}; Vd_{AUC}; Cmax; Tₘₐₓ. Results were compared between groups through ANOVA and they were graphed using the Origin 8.0 program for Windows.

Each bovine was individually weighed and dosed by SC route and bleeding was performed after administration on the following times: 15 and 30 minutes, 1, 1.5, 2, 2.5, 4, 8, 12, 24, 36, 48, 60, 72, 96, 120, 144 and 192 hours, obtaining a minimum of 10 ml per bleeding time. Samples were collected by jugular vein puncture with vacutainer tubes. Blood samples were centrifuged at 3000 rpm during 10 minutes, serum was separated and samples were kept in freezing (−4° C.) until the time of analysis which was carried out before 1 week.

Determination of Tilmicosin Serum Concentrations in Rats and Bovines:

The analytical method implemented by Bennett et al. (1966) was used. The method is described.

Agar to be used was Soy Agar Tryptic Casein (Bioxon) prepared at a rate of 40 g/l, following the indications reported in the product.

Bacterial Culture:

An ATCC (American Type Culture Collection) 25922 bacterial strain of Staphylococcus spp was used.

Bacterial Standard:

5 ml of distilled water and a sample of recent bacterial culture (re-seeded 24 hours in advance) of Staphylococcus spp. were placed in a threaded-cap tube and required adjustments to the dilution were made through Mc Farland standards in order to obtain a Mc Farland concentration of 0.5.

Turbidity at Mc Farland 0.5 was obtained by means of a spectrophotometer at 60-70% transmittance, corresponding to a bacterial concentration of 1×10⁸.

Plate Preparation:

300 ml of agar were poured and cooled for 10 minutes in a 21×20 cm Pyrex® type refractory, sterile container. 200 μl of bacterial suspension were placed over the already cold agar and was homogeneously distributed over the whole agar by means of a sterile swab.

Dilution Preparation:

20 g of tilmicosin standard (98% purity) were weighed and placed in a flask and diluted to 100 ml with deionized water (0.5 ml of a 0.1N NaOH solution was added, prior to the addition of deionized water). Ten 5 ml-tubes were marked from 1 to 10 and one of 15 ml with number 0, 9 ml of deionized water were placed in the tube numbered with 0 while 1 ml was placed into each of the remaining tubes. One ml was taken from the flask and added into tube 0 and homogenized, and 1 ml was taken therefrom and added and to tube 1 and homogenized, then 1 ml was taken from said tube and added to tube 2 and homogenized, and so on continued until completing 10 tubes, finally resulting the following dilutions:

| No. | Concentration (μg/ml) |
|---|---|
| Flask | 200 |
| Tube 0 | 20 |
| Tube 1 | 10 |
| Tube 2 | 5 |
| Tube 3 | 2.5 |
| Tube 4 | 1.25 |
| Tube 5 | 0.625 |
| Tube 6 | 0.3125 |
| Tube 7 | 0.15625 |
| Tube 8 | 0.078125 |
| Tube 9 | 0.0390625 |
| Tube 10 | 0.01953125 |

Reading of Plates:

Once the plate was prepared and with the support of a punch, two rows of 10 wells each were made along the refractory. 100 μl of each dilution were placed on each well on duplicate. 5 plates were prepared on the same day with the same methodology in order to get a total of 10 readings, which will be incubated during 24 hours at 37° C.

Readings of millimeters of halo of inhibition per well and per plate were made after 24 hours.

Reading Processing of Halos of Inhibition:

Means and standard deviation of halo of inhibition diameters were obtained for each dilution whereby, and with the support of Origin 8.0 program for Windows and Excel, the graphs of millimeters of halo of inhibition versus concentration were graphed.

Serum Processing:

Plates were prepared with the same concentration and in the same way as plates prepared to obtain a standard by tests from Bennett et al (1966); Wells were performed in the same way than the former and the same sampling times of the 10 groups were seeded in the same plate, placing 100 μl of serum, incubated during 24 hours and readings of millimeters of halo of inhibition were made, and repeated for each sampling time for every group.

Processing of Results:

Results obtained for bleeding time and those by group were extrapolated in a concentration versus halo of inhibition graph. The μg/ml of each serum sample was thus obtained.

Results:

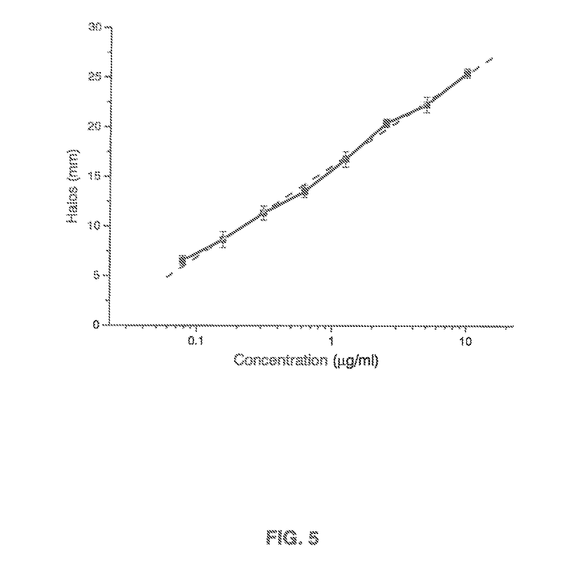

FIG. 5 of attached drawings show the analytical stage recovery curve by microbiological medium.

Average of pharmacokinetic variable means calculated by antibacterial activity composition/tilmicosin plasma concentration by means of non-compartment and compartment analysis after subcutaneously administering 2 preparations at doses of 24 mg/kg (improved extended action tilmicosin vs reference tilmicosin (Micotil®)

| | Til_{REF} | Til_{APM} | Til_{APM} (Repeat) |
|---|---|---|---|
| | (Micotil ®) | PKModel #13 | PKModel #3 |
| | model 3pk r = 98 | r = 0.98 | r = 0.96 |
| AUC_{0-∞} (μg · h/ | 100.28 | 149.62 | 203.65 |
| ml) | | | |
| AUMC (μg · h²/ | 1682.22 | 7717.46 | 14928.32 |
| ml) | | | |
| MRT (h) | 20.31 | 49.55 | 74.53 |
| T_{1/2}β (μg/ml) | 14.08 | 34.35 | 51.66 |
| T_{1/2}ab (h) | 1.11 | 6.8 | 7.01 |
| Kₑₗ (h⁻¹) | 0.05 | 0.01 | 0.01 |
| Cₘₐₓ (μg/ml) | 4.08 | 3.14 | 2.68 |
| Tₘₐₓ (h) | 3.06 | 3.88 | 0.08 |

AUC_{0-∞} = Area under the curve of concentration trapezoidal integral vs time from zero to ∞ with end stage extrapolation;
AUMC Area under the curve at first time;
MRT = Retention mean time;
α = distribution constant;
β = elimination constant rate;
Kel = Elimination constant from central compartment;
T_{1/2}ab = Absorption half-life;
T_{1/2}β = Elimination half-life;
Vd_{AUC} = AUC apparent distribution volume;
Cₘₐₓ = Maximum plasma concentration;
Tₘₐₓ = Time to achieve Cₘₐₓ

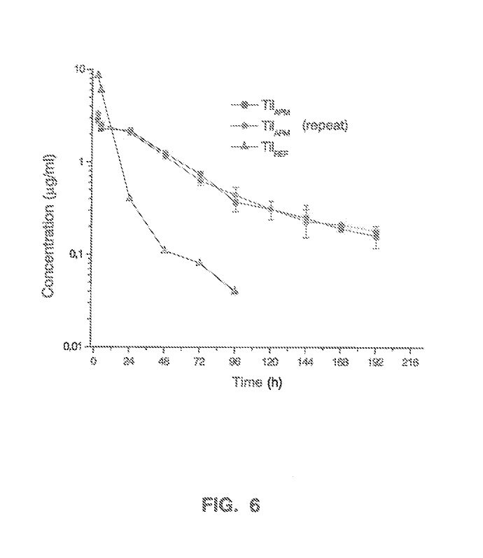

FIG. 6 of attached drawings shows the average±1 SD of preparation concentrations of tilmicosin phosphate (Til_{APM}) and compared to data achieved for reference tilmicosin (Til_{REF}).

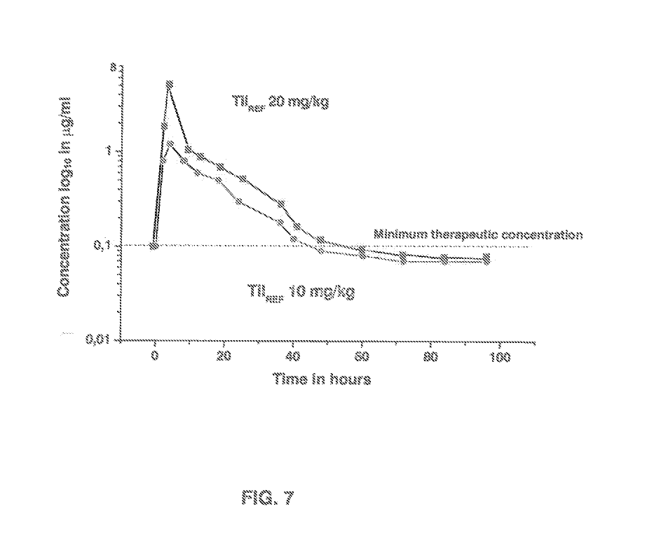

FIG. 7 of attached drawings, with the same methodology illustrated kinetic profiles of Til_{REF} dual doses as system to demonstrate that Til_{APM} does not follow a first order elimination kinetics shown by said Til_{REF} and that in fact is a system that is better described with zero-order absorption kinetics. It may be further noticed that the range remains almost unaltered.

With reference to above FIGS. 5 to 7 it is clear that:
Concentrations are raised but permanence is not increased (1st order pharmacokinetic behavior);
A higher Cmax (maximum concentration) is generated since the preparation is not designed to avoid it and is closer to the toxic critical level of tilmicosin in heart.
Til_{APM} transports peak concentrations to “stay” thus achieving useful periods in blood up to 9 days or more (note that scale is logarithmic).
It is important to notice that achieved peak is not therapeutically useful since tilmicosin is a time-dependent antibacterial drug.

In summary, substantial differences observed between Til_{APM} and Til_{REF} are:

| Til_{REF} | Til_{APM} |
|---|---|
| 48-72 hours in plasma at | At least 216 hours or |
| 10 mg/kg dose | more in plasma at 24 mg/kg |
| 72 hours in plasma at 20 mg/kg | dose |
| dose | Vehicle which prevents |
| Provides 6-10 times more | toxicity by overdosing |
| concentrations in | due to wrong calculation |
| respiratory tissue | of bovine weight |
| during 96-144 hours | Provides 6-10 times more |
| Immunostimulating for 96-144 | concentrations in |
| hours | respiratory tissue |
| Moderates inflammatory | during 432 hours (18 |
| reaction for 96-144 | days) |
| hours | Immunostimulating for 432 |
| | hours (18 days) |
| | Moderates inflammatory |
| | reaction for 432 hours |
| | (18 days) |

### Example 3

### Comparative Clinical Trial of Reference Tilmicosin (Til_{REF}) Clinical Efficacy and Improved Extended-Action Tilmicosin (Til_{APM}) in Treatment of Bovine Respiratory Disease Complex (BRD)

Objective.—

This project has the objective of comparing the clinical efficacy of Til_{REF} (Micotil®) vs. Til_{APM}, based on the premise that given that tilmicosin effect is time-dependent, any preparation of said antibacterial agent staying more than 7 days in plasma therapeutic concentrations and maybe much more in lungs (given already referred tilmicosin kinetics), may provide better clinical results in meat bovines, in defined clinical field outbreaks such as BRD.

Hypothesis.—

Intramuscular administration of Til_{APM} described and claimed in present invention, at a rate of 1 ml/15-20 kg of weight (21-28 mg/kg) elicits clinical responses higher than in empirical treatment of BRD diseases, compared to that achieved with Til_{REF} (Micotil®) at a dose of mg/kg, subcutaneously (SC) applying both on the flabby part of the neck at two sites, with a volume not higher than 7 ml per application site and in a maximum of 1 to 2 occasions.

Material and Methods.—

Mortality and morbidity percentage due to respiratory diseases fluctuates from 1.5 to 20% and from 15 to 45% respectively, therefore sample size was calculated as follows:

n=z²*p*q/d²

where:

z (confidence level)=1.96 at 95% and 2.576 at 99%

p=probability of event occurrence

q=1−p, probability of not occurring the event

d=estimated error

Therefore we have the following for this work:

n=(1.96)²*(0.45)*(1−0.45)²/(0.5)²=82 bovines

Therefore, 41 animals were at least included in each treatment (being 6 the minimum recommended on each experimental group according to USDA).

A field clinic, spontaneous, multicenter model of BRD disease presentations was used. The covered area of influence was the State of Mexico, state of Hidalgo and state of Querétaro. Bull calves Holstein, Holstein-Zebu and Holstein-Swiss were included in this study at milk production sites and on stall housing and semi-stall housing cattle.

Til_{APM} preparation was used by administering at a rate of 1 ml/15 or 20 kg weight, equivalent to a dose from 21 to mg/kg and that according to pharmacokinetic studies reaches a plasma concentration length of at least 7 days, so that a dosage range every 7 days is considered when required.

The control group as gold standard included animals treated with Til_{REF} (Micotil®) at recommended doses of 10 mg/kg every 3 days when needed. Administration was made subcutaneously on flabby part of the neck at two sites in both cases, with a volume not higher than 7 ml per application site.

Selection criteria of respiratory disease severity are shown in following Table 2:

TABLE 2
Classification by clinical signs and findings in
necropsy of three degrees of severity in Bovine Respiratory
Disease Complex.

| Clinical signs | | | |
|---|---|---|---|
| and laboratory | BOVINE RESPIRATORY DISEASE COMPLEX (BRD) | | |
| findings | GRADE I | GRADE II | GRADE III |
| Tachypnea | Rare to mild | Mild to severe | Severe |
| Dyspnea | Increase in | The same plus | The former |
| pattern | respiratory | neck | plus |
| | effort, with | stretching, | prostration |
| | chest movement | extended | and cyanoses |
| | | quarters and | |
| | | prostration | |
| Cough | Sometimes | Spontaneous | Spontaneous |
| | productive | and sporadic | and |
| | when running | and almost | continuous, |
| | | always | always |
| | | productive | productive. |
| | | | Followed by |
| | | | some lethal |
| | | | collapses |
| Rejection to | Mild or absent | Being trapped | Without |
| make effort | | almost without | showing any |
| | | effort | resistance |
| Fever | Moderate, | Above 39° C. | Always high, |
| | below 39° C. | | above 40° C. |
| Secretions | Commonly | Very commonly | Alwayas |
| through | serous or | sero-purulent | frankly |
| nostrils | purulent | | purulent |

Case assignment was randomly made, respecting the blocks as described in the table of degree of severity. Healing criterion was based on absence of fever, measured from distance by an infrared thermometer (Infrared thermometer TKTL 10, Texas Instruments, Dallas Tx, USA), taking nostrils as optical reference and when characteristic signs of degree of affectation had weakened by at least 50% upon three clinical opinions who assessed independently and treatment-blind the cases. Medication continued if the signs would not weaken 50% average on three observations.

Results are expressed in terms of quantified comparative efficacies as number of necessary injections for healing, hours to detect a reduction in basal level temperature, number of days for each occurrence to be healed and global efficacy in percentage.

Results:

Table 3 (see Attached Sheets) presents the data obtained in this multi-center trial for comparative efficacy of Til_{APM} preparation and of Til_{REF} (Micotil®), administering at a rate of 1 mL/15-20 Kg by weight in the first case (21-28 mg/kg every 7 days) or 10 mg/kg of Til_{REF} every 3 days in treatment of BRD. An statistical analysis by Chi revealed that treatment based on Til_{APM} is statistically better in variables such as: Number of treatments for healing; time to decrease basal level temperature, and this occurred in the three degrees of severity I, II and III, of disease severity (P<0.01). There was not any significant statistical difference in the number of days for healing, but it apparently required just one injection in the cases treated with Til_{APM}.

Conclusions:
Til_{APM} preparation described and claimed in the present invention is statistically more efficient compared to Til_{REF} (P<0.01) for treatment of BRD infections and including bacteria such as Actinobacillus spp., Manhemia haemolytica, Pasteurella multocida, E. coli and Haemophillus somnus, commonly associated with Mycoplasma spp.
It is important to remark that even when the two preparations are based on tilmicosin and being active vs. Mycoplasma spp., the fact that Til_{APM} preparation of present invention reaches useful plasma concentrations during 7 days and not affecting heart adds coherence to PK/PD ratio of this active principle intending a longer effect and an AUC (Area Under the Curve) value much higher than reference. Moreover, the effect of Til_{APM} preparation over Mycoplasma spp. is possibly more remarkable both for total doses and for the days that the drug remains in useful concentrations in plasma and surely in respiratory tissues.
No differences were detected between groups as to the rate whereby basal temperature is restored in affected bovines. This was as expected given that temperature reached a basal level within the time when reference tilmicosin still shows useful plasma concentrations and obviously the “improved extended-action tilmicosin” preparation also achieves suitable concentrations within the first 72 hours.
It is important to remark that most of the problems of bovine viral respiratory type [respiratory syncytial virus (RSV)], for influenza III (PI3), bovine viral rhinotracheitis (IBR) and bovine viral diarrhea (BVD), are associated with said bacteria and support treatment is common with antibacterial agents. The use for preventive effects such as in case of tulathromycin is also indicated. Therefore, conducted studies support the healing and preventive or methaphylactic use of the “improved extended-action tilmicosin” described and claimed in present invention.

### Example 4

### Use of Improved Extended-Action Tilmicosin (Til_{APM}) in Treatment of Dry Cows

Background:

Parenteral drying is established in this trial as demonstrated that specialty drugs such as tilmicosin are very well diffused throughout mammary tissue from plasma when injected subcutaneously (SC) at a 10 mg/kg dose. In fact, it is disclosed that with 48-hr tilmicosin serum concentrations achieved with Til_{REF} (Micotil®) commercial preparation, 7 days of therapeutic concentrations of tilmicosin vs. Staph. aureus are achieved.

Til_{APM} has a therapeutic serum concentration length of about 10 days therefore milk concentrations may be useful against many pathogen microorganisms, including the more difficult: Stapylococcus aureus, for at least 20 days. With the result that homogeneous concentrations may be generated without touching cow teats in the four glands or quarters of an animal when injecting a dose of improved extended-action tilmicosin at a 24 mg/kg [Til_{APM}(1)] dose and improved extended-action tilmicosin at a 30 mg/kg [Til_{APM}(2)] dose for 20 days and protection against infections in dry period is provided virtually during 45 days when injection is repeated after 20 days. Advantages are:
Treatment of 4 quarters with a single injection every 20 days (one at the beginning of dry period and another after 20 days).
Cows arriving to dry period with subclinical and even clinical infections would be treated correctly (a 40-day period of treatment) without manipulating the teats neither damaging meatus nor teat channel. Fibrous teat obstruction would not be broken.
Having described that most of clinical mastitis during lactation is acquired during the dry period, a good dry period is congruent to be thought to reflect a lower rate or incidence of clinical mastitis during the first 8 weeks, when animals are more susceptible.
That above is translated into a higher milk production during the lactation period (one case of mastitis reduces milk producing mammary tissue up to 50%).

Hypothesis:

Til_{APM} (1) and Til_{APM} (2) administration upon commencement of dry period and 20 days after, generates sufficiently high concentrations in milk or mammary fluid (obtained from teat) to be considered as therapeutic during the whole dry period (40-45 days).

Objectives:

1. Determining tilmicosin concentrations in milk samples or mammary fluid of cows treated SC with Til_{APM} (1) and Til_{APM} (2), applying at the beginning of lactation and 20 days later.

2. Assessing the mastitis rate in several herds where said Til_{APM} scheme is applied on the first 8 weeks after labor.

3. Assessing a response in microbiological terms in cows with high somatic cell counts (CCS) at the beginning of lactation and/or when being positive to Staphylococcus aureus or Streptococcus sp., one week after labor.

Material and Methods:

Holstein/Friesian cows proceeding from 5 establishments dedicated to milk production in Tlaxcala and State of Mexico were only used for this assay

### Stage I. Determination of Tilmicosin Concentrations in Mammary Fluids During Dry Period and Activity Against Staphylococcus aureus

A total of 32 cows were included in this study applying the dry period procedure of a SC route dose of Til_{APM} (1) and Til_{APM} (2) SC upon starting the dry period and 20 days later. These animals had an average production from previous cycle of 23.8±5.6 L and all were in the list of possible disposals as being positive to Staphylococcus aureus before dry period and given their history of multiple mastitis relapses in spite of a large variety of treatments with any type of intramammary antibacterials. All animals showed values of 2 or 3 in California test and somatic cell values >1,000,000 before drying.

Cows were allowed concluding their dry period, but animals were only sampled once by initial mild massage, injection of 0.5 UI/kg of weight of intramammary (IM) oxytocin and milking of at least 350 ml, wherein the first 100 ml were disposed in order to achieve a more representative sample of gland tissue from deep mammary tissue and not only from cisternal residual.

Samplings were conducted in such way to meet 45 days of dry period with three animals per time, thus:

| Total cows | Dry period days |
|---|---|
| 4 | 3 |
| 4 | 7 |
| 4 | 14 |
| 4 | 20 before second |
| | injection |
| 4 | 23 |
| 4 | 30 |
| 4 | 37 |
| 4 | 44 |

Furthermore, colostrum samples were taken within 2 to 3 hours after labor and Delvotest® tests were conducted in order to assess if there was any presence of inhibiting agents.

In order to assess microbiologically all cows in this section of the trial, milk samples were taken on days 7, 14 and 21, and a bacteriological analysis with emphasis on Staphylococcus aureus detection was made, performing growths in mannitol salt agar (Chapman medium) as a way to isolate said bacterium.

Determination of Tilmicosin in Milk Samples:

This was performed by high resolution chromatography according to the method disclosed in residue stage. The procedure includes extraction of milk samples with acetonitrile and cleaning with Sep-Pack cartridges (Waters, USA).

### Stage II. Determination of Mastitis Rate 45 Days after Labor with Parenteral Dry Cow Based on Til_{APM}

Five cowsheds from the states of Tlaxcala and State of Mexico with similar technification characteristics participated in this part of the trial: mechanic milking, tipping before milking and cleaning and sealing thereafter, two milkings and clean facilities at milking room. Moreover, assessments of California test to all animals at least twice per week were performed and somatic cell counts in tank, daily and in suspected cows when required were available. Cows were dried for 45 days in all, these cowsheds and a single intramammary tube was used per room. Beta-lactams, including cephapirin benzathine, neomycin, and the like were used at these sites. About 20 to 35 dry cows per month were present. Then, animals were randomly separated in conventional treatment (with intramammary tube) and experimental treatment with parenteral Til_{APM} at a dose reported of 24 and 30 mg/kg upon dry period commencement and another injection 20 days later. General characteristics of involved herd sizes were as follows:

| | | | No. cows | |
|---|---|---|---|---|
| | | No. cows | treated with | No. cows |
| | No. cows in | dried per | Til_{AMP} in this | treated |
| Cowshed | production | month | trial | conventionally |
| El Puente | 1,500 | 30 | 30 | 45 |
| Rancho | 750 | 22 | 18 | 26 |
| Grande | | | | |
| El Sauzal | 2,000 | 48 | 32 | 30 |
| Sta Mónica | 1200 | 28 | 35 | 23 |
| La Curva | 1650 | 35 | 22 | 18 |

All, animals included in this stage were admitted clinically healthy to this trial, with a somatic cell count lower than 300,000 CS/mL as California negative inclusion criterion and without positive bacteriological data.

Two parameters were assessed: incidence of clinical mastitis post-labor up to 8 weeks (56 days later), without registering severity or etiology. A count of individual somatic cells from cows under study was further made through an automatic counter (DeLaval Cell counter DCC, USA).

Results:

Stage I.—

Table 4 shows tilmicosin concentrations found in cows treated with Til_{APM} (1) at a rate of 24 mg/kg SC upon commencement of dry period and 20 days later.

TABLE 4
Mean values ± SD of tilmicosin concentrations in
milk or milk fluid extracted during Holstein/Friesian dry cow
after two Til_{APM} administrations at doses of 24 mg/kg on day
zero and on day 20. Each point is mean ± SD of 4 samples

| Days of dry | Mean of four | |
|---|---|---|
| period | determinations | ±1 SD |
| 3 | 12.2 | 3.5 |
| 7 | 8.4 | 2.6 |
| 14 | 3.8 | 2.2 |
| 20 | 1.2 | 0.6 |
| 23 | 13.5 | 3.9 |
| 30 | 9.2 | 2.8 |
| 37 | 4.1 | 1.2 |
| 44 | 0.8 | 0.6 |

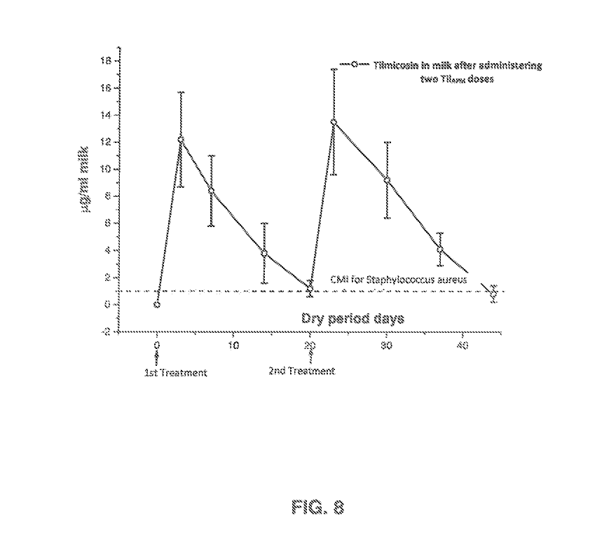

FIG. 8 of attached drawings shows tilmicosin concentrations in milk or milk fluid extracted during dry period of Holstein/Friesian cows, after two tilmicosin administrations of Til_{APM} (1) at a dose of 24 mg/kg on day 0 and on day 20. Each point is the mean±1 of 4 samples. CMI value for most of Staphylococcus aureus is <1.0 μg/mL.

Bacteriological analyses to find Staphylococcus aureus revealed that just 5 samples out of 32 assessed on day 7 were positive for this pathogen (84.37% free of Staph. aureus). On day 14, 6 cows were positive to Staph. aureus (81.25.% free of Staph. aureus). On day 21 no variation was detected regarding day 14. On the contrary, there was no healing in the group treated conventionally and all cows resulted positive to Staphylococcus aureus one week after labor.

All samples of cows treated with Til_{APM} resulted negative for Delvotest® test after labor and all tests made on samples obtained during dry period were positive.

With data obtained and the number of treated glands and a control group considered as single treatment (conventional) a product efficacy of at least 80% was achieved assuming a control group response of 0.01%; resulting a P=0.001 value and a test potency=0.95 (GPower).

Stage II.—

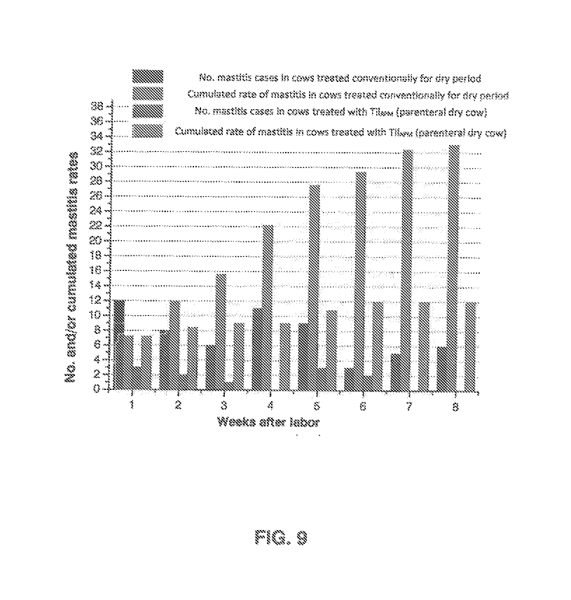

Results of this stage developed along 14 months are shown as percentage in cumulated and summed mastitis rate of 5 sites both for cows treated with Til_{APM} (167 cows) under already explained mode and cows treated conventionally (142 cows) and is illustrated in FIG. 9 of attached drawings, where the number of mastitis cases per week post-labor is shown, for cows treated upon dry period starting with intramammary antibiotics of varied nature and registered for such use (conventional treatment) and cows treated parenterally with Til_{APM} (1) at a subcutaneously (SC) administered dose of 24 mg/kg when starting the dry period and 20 days later.

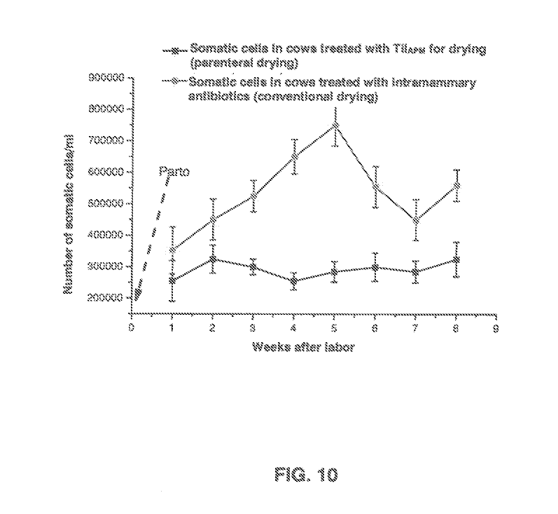

FIG. 10 of attached drawings shows trends in somatic cell counts in milk from cows treated upon starting dry period with intramammary antibiotics of various nature and registered for such use (conventional treatment), and cows treated parenterally with Til_{APM} (1) at a dose of 24 mg/kg upon starting dry period and subcutaneously (SC) 20 days later.

Conclusions:

Treatment of dry cows with a Til_{APM} (1) scheme at subcutaneous (SC) doses of 24 mg/kg upon starting this period and 20 days later generates a healing higher than 80% in cows with Staphylococcus aureus infection. This value is higher than those found in literature fluctuating in most cases around 30% of healing.

That above clearly reflects a significant decrease in post-labor mastitis rate which is perfectly in agreement with a low somatic cell incidence and being apparently known that an increase in cell count is closely related to a higher percentage of cows with udder (subclinical and clinical) problems. Subclinical and obviously clinical mastitis affect milk producing tissue and causes a decrease in production.

### Example 5

### Study on Residue Depletion of Tilmicosin from Improved Extended Action Tilmicosin Preparation (Til_{APM})

Center of Sample Analysis:

Chromatography: Research Laboratory 2317 of Physiology and Pharmacology Department belonging to the Faculty of Veterinary Medicine and Animal Husbandry of the National Autonomous University of Mexico.

Study Objective:

Study on residue depletion and local tolerance to Til_{APM} veterinary medicament in bovines.

Regulatory Guidelines:

Vich guidelines gl43, gl48

Identification of Trial Product:

Til_{APM} injectable solution at 20%

Composition:

| | |
|---|---|
| Til_{APM} | 20 g |
| Vehicle c.b.p | 100 ml |

Administration and Posology:

Subcutaneously (SC) administered on flabby part of the neck without exceeding a limit of 10 ml per site at a 30 mg/kg rate of Til_{APM} product (2).

Experimental Design:
Animals: 11 bull calves F1 Holstein/Zebu with average initial weight of 276.09±37.15 kg were used, proceeding from Rancho “El Clarín” at Martinez de la Torre, Veracruz, belonging to the Faculty of Veterinary Medicine of National Autonomous University of Mexico and the absence of any other type of medication was confirmed through ranch registrations and blood basal sampling from animals. Records confirmed that animals did not receive any drug 45 days before starting this trial.
Inclusion and Non-inclusion Criteria: Animals showed a weight range from 180 to 235 kg (average animal weight was 276.09±37.15 kg) and they did not receive any medication 45 days before starting this trial. Bull calves did not show any sign of disease and they were F1 Holstein/Zebu. Animals showing any respiratory or digestive sign were not included as well as those treated with any antibacterial or analgesic within a margin of 1 month.
Exclusion criteria: Those animals which did not meet already mentioned inclusion criteria. If any animal were positive on basal blood samples at tilmicosin base it would be excluded from testing.
Identification: Animals included in the test were identified with special cattle earrings with animal number. Each animal was individually weighed and dosed. Table 5 shows the weight and individual dosage in grams of product and administered volume.

TABLE 5
Weight of animals individually used and dosed with
Til_{APM} veterinary product, injectable solution at 40% and a 30 mg/kg dose

| Animal | Peso (kg) | Dosis (g/animal) | Dosis (mL/animal) |
|---|---|---|---|
| 1 | 258 | 6708 | 16.77 |
| 2 | 269 | 6994 | 17.485 |
| 3 | 258 | 6708 | 16.77 |
| 4 | 189 | 4914 | 12.285 |
| 5 | 325 | 8450 | 21.125 |
| 6 | 265 | 6890 | 17.225 |
| 7 | 301 | 7826 | 19.565 |
| 8 | 321 | 8346 | 20.865 |
| 9 | 295 | 7670 | 19.175 |
| 10 | 278 | 7228 | 18.07 |
| 11 | 278 | 7228 | 18.07 |

A maximum of 10 ml of product was injected per subcutaneous injection point on the flabby part of the neck and the point where the highest volume is applied was then marked in order to obtain tissues which were used for residue analysis.

Adaptation and Animal Housing:

Animals were housed in their common environment with food and water ad libitum.

Diet and Housing:
Fodder: Animals under testing received the usual common diet at the facility and formulated according to their requirements for development and production.
Water: All animals received water ad-libitum and suitable for its consumption
Housing: Animals were housed in pens under semi-intensive conditions

Environmental Conditions:

Martínez de la Torre is located in Northern Veracruz, at coordinates 20° 04′ North latitude and 97° 04′ West longitude, at a height of 151 meters above sea level. Its climate is regular humid-hot with an average temperature of 23.7° C. Average annual rain fall is 1,293.6 millimeters.

Treatment:

Til_{APM} veterinary product was applied at 40%.

Drug Administration:

Calculated dose for each animal was subcutaneously administered. Dose was 1 ml/15-20 kg weight/day (equivalent to 24 mg of Tilmicosin base/kg weight), without administering more than 10 ml per application site. A graded syringe not larger than 10 ml was used for drug administration so that the most accurate possible dose was kept and an 18-gauge needle was used.

The final Til_{APM} product fraction which was not used was treated as residue and disposed according to chemical hazardous waste management provided by the Faculty of Veterinary medicine of National Autonomous University of Mexico, pursuant to regulation provision NOM-052-058 SEMARNAT-2005.

Sacrifice and Sampling:
Preparation method and sample processing: With T½β (elimination half-life) data obtained in kinetic phase of this product, a projection to zero residues was made in 10 bull calves, and three different sacrifice days and sampling were present: one before the time of zero residues and two later to demonstrate the presence and later absence of residues of the following way;

log C=C(0)−kt²⁴ Formula:

where
C=concentration
C(0)=concentration on time zero by regression straight line extrapolation
k=α, β or γ
t=time

Sample Preparation and Conservation:

Injection point was accurately determined after administration by shaving (15 cm around the point), allowing later assessment of preparation local tolerance and sampling. Animals were daily monitored to detect and to register possible immediate and mediate adverse reactions. Animals were sacrificed at the slaughterhouse (3 or 4 per day). Samples from 10 to 50 g were obtained from the following animal organs: kidney, liver, muscle (administration site) and perirenal fat, tagged and kept frozen at −20° C. until the time of analysis (no more than 7 days from sampling). One of the bull calves was used as control, to which the treatment was not applied. Carcasses of sacrificed animals were burned.

Sample Submission and Transportation:

Samples of kidney, liver, muscle (injection point) and perirenal fat were kept frozen at −20° C. until the time of analysis.

Sample Analysis:
Sample reception and conservation: Samples were received frozen by means of liquid nitrogen. These were identified and each sample separated in two vials and kept frozen until their analysis by chromatography, which was no later than 30 days after sample collection.
Analysis of Laboratorios Karizoo Til_{APM} residues: A modified method was used for tilmicosin base residue analysis based on the method developed by Stobba-Wiley et al. (2000), whereby a recovery percentage from 80 to 85% and a reproducibility coefficient with a 5% variation was achieved.
Sample preparation: 5 grams of tissue were weighed in a Falcon type polypropylene tube and 10 ml of acetonitrile were added, mixed during 10 minutes at 100 rpm with a rotating stirrer. 10 ml of isooctane were added and mixed during 5 minutes at 30 rpm. (Preventing that emulsion is formed). That was then centrifuged during 10 minutes at 4000 rpm. Top phase (isooctane) was removed and 8 ml of lower phase was pipetted and diluted with 50 ml of deionized water.

50 ml were placed in a reservoir and connected to a SPE Bond Elut C18 cartridge (6 ml), which was activated with 1 ml of dimethyl dichlorosilane followed by 10 ml of methanol and 10 ml of deionized water. Extracted sample was obtained by a flow of 2 drops. Elution was poured into a 10 ml tube and reconstituted with 1 ml of acetonitrile. Obtained sample was then prepared according to the technique described in pharmacokinetics.

Samples of free target tissue (1 g for liver, kidney, muscle and fat) obtained from untreated animal were added with tilmicosin base standard solutions using 10 μl of each solution to obtain final concentrations of 1, 5; 10; 20, 40, 80 ng of tilmicosin base/g of tissue and final concentrations of 1, 5; 10; 20; 40, 80 ng. Following the samples of liver, kidney, muscle and fat added with tilmicosin base solutions, 0.5 ml of deionized water and 2 ml of acetonitrile were added and stirred in a mixer (Vortex) for 20 minutes, then micronized in an ultrasound bath for 20 minutes followed by sample centrifugation at 4000 rpm for 10 minutes.

Results:

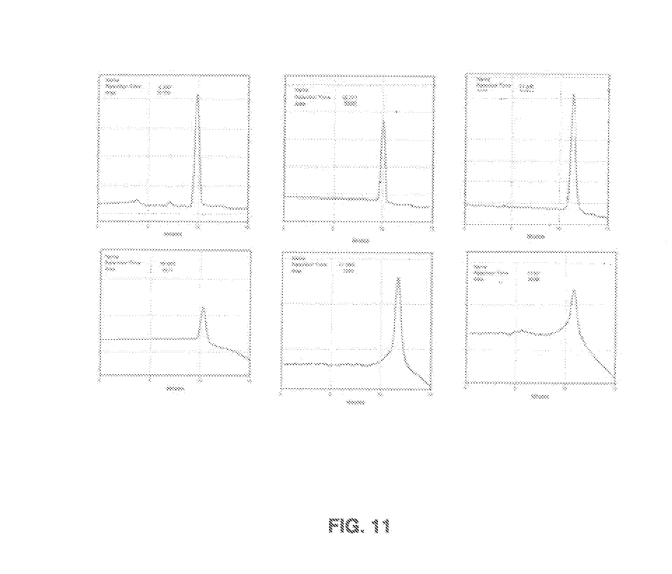

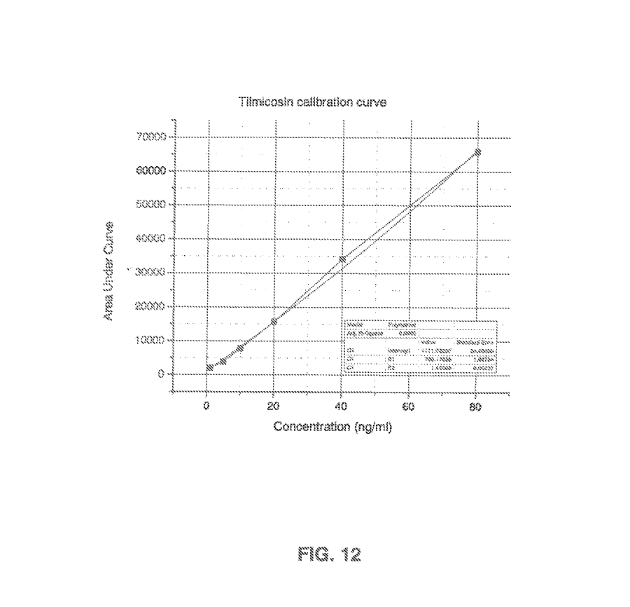

All animals were negative in blood basal samples therefore no animal was excluded from the protocol. Table 6, as well as FIGS. 11 and 12 in attached drawings, illustrate retention peaks of areas under the curve of tilmicosin base standard concentrations by means of HPLC. By an extrapolation of the kinetic study elimination stage and with the formula detailed in sacrifice paragraph and sampling, the following sacrifice times were determined:
Before the day of “zero residues” (10 days)
Day of “zero residues” (30 days)
After day of zero residues (35 days)

Tables 7 to 10 illustrate recovery percentages of tilmicosin base in liver, kidney, muscle, fat respectively and administration site.

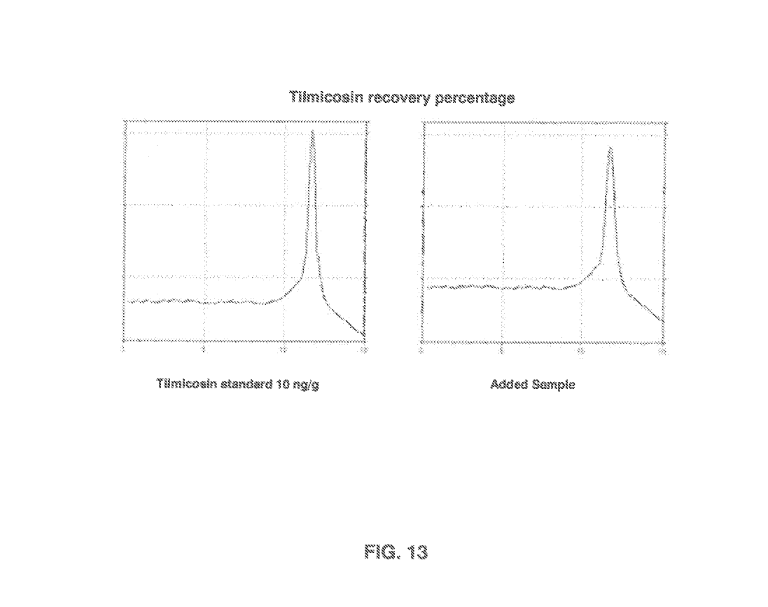

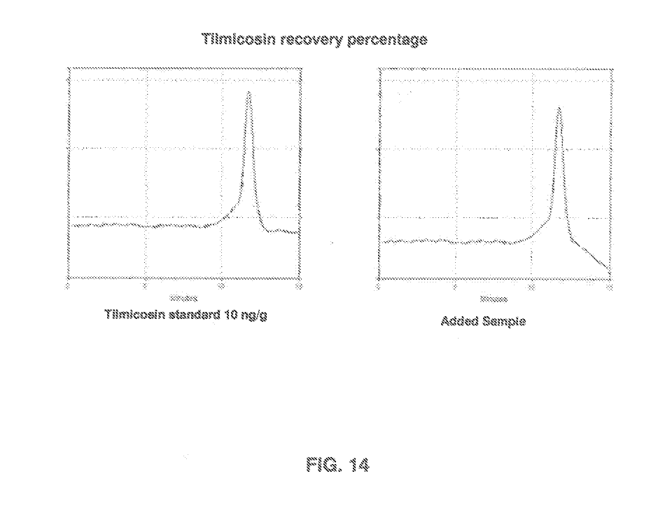

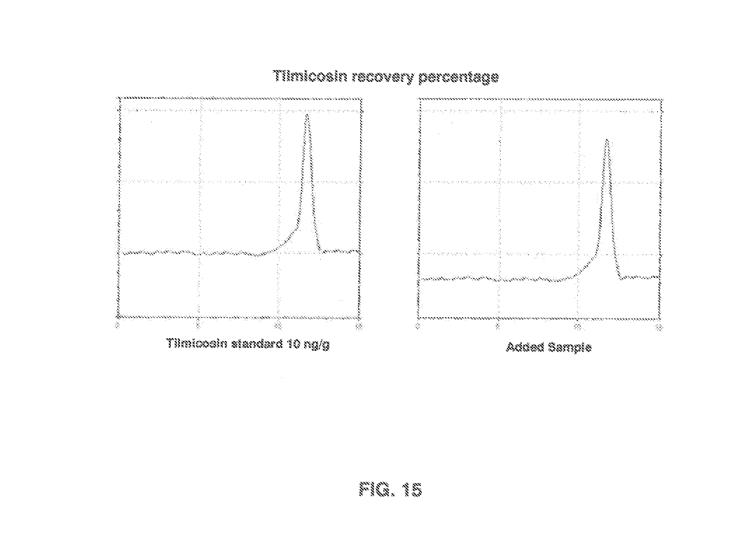

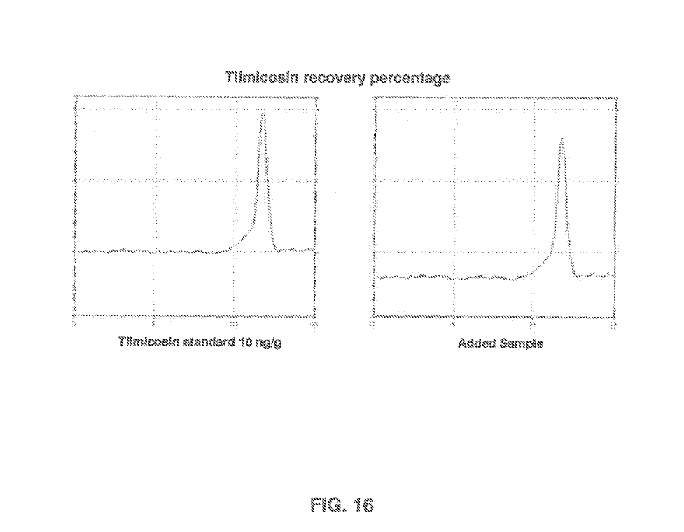

FIGS. 13 to 16 in attached drawings show an example of tilmicosin base standard and of each fortified tissues.

Tables 11 to 14 show the concentrations obtained in liver, kidney, muscle and fat per animal per sampling day and FIG. 11 shows the tilmicosin base concentrations found in all studied tissues. While table 15 shows the concentrations on application site.

TABLE 6
Area Under the curve (average) of calibration
curve obtained from HPLC reading

| Concentration | Average Area Under | |
|---|---|---|
| ng/mL) | Curve* | SD± |
| 80 | 65922 | 245.4 |
| 40 | 37191 | 489.3 |
| 20 | 15692 | 6.2 |

Analysis was made in triplicate

TABLE 7
Tilmicosin base recovery percentage in liver fortified
with different drug concentrations

| | | Average | |
|---|---|---|---|
| Fortified | Average | fortified | |
| concentration | standard area | liver* area | Recovery |
| (ng/g) | under curve | under curve | percentage |
| 20 | 12692 | 10788 | 85 |
| 10 | 6281 | 5339 | 85 |
| 5 | 3048 | 2530 | 83 |
| 1 | 1571 | 1257 | 80 |

*Analysis was made in triplicate

TABLE 8
Tilmicosin base recovery percentage in kidney
fortified with different drug concentrations

| | | Average | |
|---|---|---|---|
| Fortified | Average | fortified | |
| concentration | standard area | kidney* area | Recovery |
| (ng/g) | under curve | under curve | percentage |
| 20 | 12492 | 10618 | 85 |
| 10 | 6292 | 5285 | 84 |
| 5 | 3128 | 2628 | 84 |
| 1 | 1651 | 1321 | 80 |

*Analysis was made in triplicate

TABLE 9
Tilmicosin base recovery percentage in fortified
muscle with different drug concentrations

| | | Average | |
|---|---|---|---|
| Fortified | Average | fortified | |
| concentration | standard area | muscle* area | Recovery |
| (ng/g) | under curve | under curve | percentage |
| 20 | 12221 | 10387.85 | 85 |
| 10 | 6156 | 5232.6 | 85 |
| 5 | 3101 | 2573.83 | 83 |
| 1 | 1555 | 1275.1 | 82 |

*Analysis was made in triplicate

TABLE 10
Tilmicosin base recovery percentage in fortified fat
with different drug concentrations

| | | Average | |
|---|---|---|---|
| Fortified | Average | fortified | |
| concentration | standard area | liver* area | Recovery |
| (ng/g) | under curve | under curve | percentage |
| 20 | 12110 | 10051.3 | 83 |
| 10 | 6258 | 5194.14 | 83 |
| 5 | 3100 | 2542 | 82 |
| 1 | 1472 | 1177.6 | 80 |

*Analysis was made in triplicate

TABLE 11
Tilmicosin base concentrations (ng/g) in liver

| Day | Animal 1 | Animal 2 | Animal 3 | Average | ±SD |
|---|---|---|---|---|---|
| 10 | 7.21 | 8.54 | 8.02 | 7.92 | 0.67 |
| 30 | 0.91 | 0.72 | 0.54 | 0.72 | 0.19 |
| 35 | Nd | Nd | Nd | Nd | nd |

TABLE 12
Tilmicosin base concentrations (ng/g) in kidney

| Day | Animal 1 | Animal 2 | Animal 3 | Average | ±SD |
|---|---|---|---|---|---|
| 10 | 8.12 | 9.23 | 8.57 | 8.64 | 0.56 |
| 30 | 0.21 | 0.23 | 0.34 | 0.26 | 0.07 |
| 35 | Nd | Nd | Nd | Nd | nd |

TABLE 13
Tilmicosin base concentrations (ng/g) in muscle

| Day | Animal 1 | Animal 2 | Animal 3 | Average | ±SD |
|---|---|---|---|---|---|
| 10 | 6.23 | 3.42 | 7.21 | 5.62 | 1.96 |
| 30 | 0.22 | 0.28 | 0.21 | 0.23 | 0.03 |
| 35 | Nd | Nd | Nd | Nd | nd |

TABLE 14
Tilmicosin base concentrations (ng/g) in fat

| Day | Animal 1 | Animal 2 | Animal 3 | Average | ±SD |
|---|---|---|---|---|---|
| 10 | 6.23 | 5.18 | 5.53 | 5.70 | 0.53 |
| 30 | 0.75 | 0.54 | 0.37 | 0.55 | 0.19 |
| 35 | Nd | Nd | Nd | Nd | nd |

TABLE 15
Tilmicosin base concentrations (ng/g) on application site

| Day | Animal 1 | Animal 2 | Animal 3 | Average | ±SD |
|---|---|---|---|---|---|
| 10 | 9.23 | 12.1 | 7.28 | 9.53 | 2.42 |
| 30 | 0.85 | 0.93 | 0.73 | 0.84 | 0.10 |
| 35 | Nd | Nd | Nd | Nd | nd |

### Protocol Modifications:

Not provided.

Sample Conservation and Documentation:

Samples were identified and stored in cryovials and Ziploc® bags and kept under freezing (−20° C.) until the time of analysis. Final destruction of samples was performed by treatment as pathological wastes according to waste management established in the Faculty of Veterinary Medicine of the National Autonomous University of Mexico, pursuant to regulation provision NOM-052-058 SEMARNAT-2005, therefore those were incinerated.

Final Report (Conclusions):
Elimination half-life values indicate that tilmicosin base injectable product (20% injectable solution) reaches zero theoretical concentrations around day 30 so that waste depletion analysis times are:
   Before zero residue day (10 days)
   The day of presence of zero residues (30 days)
   After the hypothetic day of zero residues (35 days)

It was demonstrated that animals had not been previously medicated, obtaining zero residues in the day prior to administering the product to be assessed and residues were found in some target tissues on day 30^{th}; on day 35^{th} no residues were found in any of the assessed tissues. Therefore, and taking into consideration the attachment suggestions, a withdrawal time from 35 to 40 days after the last application is proposed.

Even when above description has been referred to certain embodiments of the improved extended-action tilmicosin of present invention, it should be remarked that several modifications to such embodiments are possible, but without deviating from the true scope of the invention. Therefore, the present invention should not be restricted except for the provisions in the state of the art and by the attached claims.

1. A new tilmicosin formulation having the formula 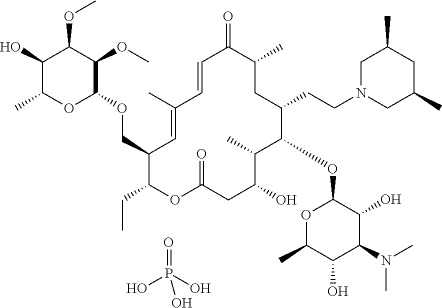
wherein said tilmicosin has been formulated for improved extended action or release.

2. The tilmicosin formulation according to claim 1, wherein the release is extended from 9 to 10 days in plasma.

3. The tilmicosin formulation according to claim 2, wherein said formulation comprises: tilmicosin phosphate in a concentration from 35 to 50% by weight of total composition; a first co-solvent in a concentration from 8 to 20% by volume, wherein said first co-solvent is selected from the group consisting of sorbitol, glycerin, glyceride ester derivatives, ethyl alcohol, isopropyl alcohol, propylene glycol, benzyl alcohol, low molecular weight polyethylene glycol (100, 200, 300, 400, 600 and 800) and polyethylene glycol derivatives, dimethyl sulfoxide, glycerol formal, glycofurol, ethyl carbonate, ethyl lactate, dimethyl acetamide or 2-methyl pyrrolidone, and the like; a second co-solvent in a concentration from 5 to 15% by volume, wherein said second co-solvent is selected from the group consisting of sorbitol, glycerin, glyceride ester derivatives, ethyl alcohol, isopropyl alcohol, propylene glycol, benzyl alcohol, low molecular weight polyethylene glycol (100, 200, 300, 400, 600 and 800) and polyethylene glycol derivatives, dimethyl sulfoxide, glycerol formal, glycofurol, ethyl carbonate, ethyl lactate, dimethyl acetamide or 2-methyl pyrrolidone, and the like; and, an emulsifier in a concentration from 1.5 to 15% by weight of total composition, wherein said emulsifier may be any surfactant with emulsifying activity capable of stabilizing the system.

4. The tilmicosin formulation according to claim 3, wherein tilmicosin phosphate is in a concentration of 42% by weight of total composition.

5. The tilmicosin formulation according to claim 3, wherein the first co-solvent is in a concentration of 10% by volume.

6. The tilmicosin formulation according to claim 5, wherein the first co-solvent is propylene glycol.

7. The tilmicosin formulation according to claim 3, wherein the second co-solvent is in a concentration of 10% by volume.

8. The tilmicosin formulation according to claim 7, wherein the second co-solvent is ethyl alcohol.

9. The tilmicosin formulation according to claim 3, wherein the emulsifier is in a concentration from 3% by weight of total composition weight.

10. The tilmicosin formulation according to claim 9, wherein the emulsifier is poloxamer.

11. A method of preparation of an improved extended-action tilmicosin formulation according to precedent claim 1, comprising the stages of:
(a) Transferring from 45 to 60 ml of distilled water to a beaker;
(b) Adding to the beaker containing distilled water an amount from 8 to 20 ml of a first co-solvent selected from the group consisting of sorbitol, glycerin, glyceride ester derivatives, ethyl alcohol, isopropyl alcohol, propylene glycol, benzyl alcohol, low molecular weight polyethylene glycol (100, 200, 300, 400, 600 and 800) and polyethylene glycol derivatives, dimethyl sulfoxide, glycerol formal, glycofurol, ethyl carbonate, ethyl lactate, dimethyl acetamide or 2-methyl pyrrolidone, and the like; and stirring until obtaining a homogeneous solution;
(c) Adding an amount from 5 to 15 ml of a second co-solvent selected from the group consisting of sorbitol, glycerin, glyceride ester derivatives, ethyl alcohol, isopropyl alcohol, propylene glycol, benzyl alcohol, low molecular weight polyethylene glycol (100, 200, 300, 400, 600 and 800) and polyethylene glycol derivatives, dimethyl sulfoxide, glycerol formal, glycofurol, ethyl carbonate, ethyl lactate, dimethyl acetamide or 2-methyl pyrrolidone, and the like; and covering the beaker with parafilm paper mixing until obtaining a homogeneous solution;
(d) Introducing slowly an amount from 35 to 50 g of tilmicosin phosphate, in order to prevent lump formation, and stirring until obtaining a homogeneous solution;
(e) Adding one from 1.5 to 15 g of an emulsifier, wherein said emulsifier may be any surfactant with emulsifying activity capable of stabilizing the system, making a slow addition to achieve solution homogenization;
(f) Refrigerating above solution at a temperature from 2 to 8° C. during 1 hour, removing from refrigeration and stirring by 5 minutes;
(g) Refrigerating again for 24 hours stirring until obtaining a full dissolution of poloxamer, thus obtaining the final product.

12. The method of preparation of an improved extended-action tilmicosin formulation according to claim 10, wherein the transferred amount of distilled water is 53 ml.

13. The method of preparation of an improved extended-action tilmicosin formulation according to claim 10, wherein the first co-solvent is added in a volume of 10 ml.

14. The method of preparation of an improved extended-action tilmicosin formulation according to claim 13, wherein the first co-solvent is propylene glycol.

15. The method of preparation of an improved extended-action tilmicosin formulation according to claim 10, wherein the second co-solvent is added in a volume of 10 ml.

16. The method of preparation of an improved extended-action tilmicosin formulation according to claim 15, wherein the second co-solvent is ethyl alcohol.

17. The method of preparation of an improved extended-action tilmicosin formulation according to claim 10, wherein tilmicosin phosphate is added in an amount of 42 g.

18. The method of preparation of an improved extended-action tilmicosin formulation according to claim 10, wherein the emulsifier is added in an amount of 3 g.

19. The method of preparation of an improved extended-action tilmicosin formulation according to claim 18, wherein the emulsifier is poloxamer.

20. (canceled)

21. A method of treating or prophylaxis of bacterial type respiratory infectious diseases, such as Bovine Respiratory Disease Complex (BRD) and “dry cow” period in a mammal comprising administering the improved extended-action tilmicosin formulation according to claim 1 to the mammal in need thereof.
